(19) United States
(12) Patent Application Publication
(10) Pub. No.: US 2019/0269549 A1
(43) Pub. Date: Sep. 5, 2019

(54) THERMOTHERAPY EQUIPMENT

(71) Applicant: CERAGEM CO., LTD, Cheonan-si (KR)

(72) Inventors: Keun Young PAEK, Cheonan-si (KR); Sang Ho CHOI, Cheonan-si (KR); Seung Gwan HONG, Cheonan-si (KR); Dong Myoung LEE, Asan-si (KR); Jin Cheol PARK, Cheonan-si (KR)

(73) Assignee: CERAGEM CO., LTD, Cheonan-si (KR)

(21) Appl. No.: 16/334,108

(22) PCT Filed: Mar. 23, 2017
(86) PCT No.: PCT/KR2018/003454

§ 371 Date: Mar. 18, 2019

(30) Foreign Application Priority Data
Mar. 23, 2017 (KR) 10-2017-0036911

(51) Int. Cl. A61F 7/08; A61H 9/00

(52) U.S. Cl. CPC A61F 7/08; A61F 2007/0024; A61H 9/0071

(57) ABSTRACT

The present invention relates to a thermotherapy bed, and more specifically, to a slide-type thermotherapy bed, of which an air massage module is easy to store and use and which is used in thermotherapy. The thermotherapy bed of the present invention includes a body in which a thermotherapy device is installed, an auxiliary unit sliding on the body to be unfolded or overlap the body, such that the thermotherapy device is opened or closed, a mat configured to cover an upper portion of the auxiliary unit, and an air massage module installed in the mat and stored in the auxiliary unit.

### CROSS-REFERENCE TO RELATED APPLICATIONS

This application is a national phase application under 35 U.S.C. § 371 of International Application No. PCT/KR2018/003454, filed Mar. 23, 2018, which claims priority to and the benefit of Korean Patent Application No. 2017-0036911, filed on Mar. 23, 2017, the disclosures of which are incorporated herein by reference in their entirety.

### FIELD OF THE DISCLOSURE

The present invention relates to a thermotherapy bed, and more specifically, to a slide-type thermotherapy bed, of which an air massage module is easy to store and use and which is used in thermotherapy.

### DESCRIPTION OF RELATED ART

Conventionally, in order to relieve acute or chronic pain in muscle and nervous tissues of a vertebral part generated due to long, continuous work in an improper posture and habituation of the inappropriate posture, to improve blood circulation of a body, and to relieve instantaneous muscle stitch, a thermotherapy bed has been widely used to improve blood circulation by providing a heat stimulus to a pain generation site while moving along a body part.

Since existing thermotherapy beds used in such thermotherapy are formed in the form of a general non-folding bed. Although the existing thermotherapy beds are not used, the existing thermotherapy beds occupy large spaces.

When an air massage is performed with thermotherapy, there is a cumbersome problem in carrying and storing of an air massage device.

Since an air pump and a hose for supplying air to an air pocket are exposed to the outside, there is a problem with aesthetics thereof.

The present invention is directed to a slide-type thermotherapy bed, of which an air massage module is easy to store and use and which is used in thermotherapy.

### SUMMARY OF THE INVENTION

According to an aspect of the present invention, there is provided a thermotherapy bed including: a body in which a thermotherapy device is installed; an auxiliary unit sliding on the body to be unfolded or to overlap the body, such that the thermotherapy device is opened or closed; a mat configured to cover an upper portion of the auxiliary unit; and an air massage module installed in the mat and stored in the auxiliary unit.

The auxiliary unit may have a base portion sliding on an upper portion of the body, and a movement portion extending downward from an end of one side of the base portion.

The base portion may have a module storage portion for storing the air massage module.

The module storage portion may include a sensor configured to determine whether the air massage module is stored.

The air massage module may be stored in the module storage portion and may not be exposed to the outside, and subsequent to when a user uses the thermotherapy bed, the mat may be partially folded, such that the air massage module is exposed to the outside from the module storage portion.

The air massage module may include a plate portion installed on a lower portion of the mat and air pockets installed on the plate portion.

The air pockets may have first and second air pockets disposed at both sides of a fixing portion installed on the plate portion.

The first and second air pockets may include a plurality of air cells which are vertically stacked.

A user's calves may be disposed at the fixing portion, and the first and second air pockets may be expanded into a fan shape with respect to the fixing portion by air supplied from an air pump and surround the user's calves.

Detachable portions may be formed at ends of both sides of the air pocket.

The air pocket may include an extension gender attached to and detached from the detachable portions.

According to the thermotherapy bed according to the present invention, an arrangement space can be further secured through an auxiliary unit sliding on a body to be unfolded or to overlap the body.

In addition, when the auxiliary unit slides to overlap the body, the thermotherapy bed can be used as a heating sofa (stool).

Further, when the auxiliary unit slides from the body to be unfolded and the thermotherapy bed is used as a heat massage device, the user can select and use an air massage module and a thermotherapy module of the auxiliary unit.

In addition, a calf massage can be easily performed through the air massage module and a module storage unit, and an easy storage effect can be obtained.

Furthermore, an air pump and a hose can be accommodated inside the thermotherapy bed, thereby obtaining an effect of enhanced aesthetic sense.

In addition, in consideration of different positions of calves according to the users, an arrangement position of the air massage module can be adjusted.

### BRIEF DESCRIPTION OF THE DRAWINGS

FIG. 1 is a perspective view illustrating a thermotherapy bed according to a first exemplary embodiment of the present invention;

FIGS. 2 and 3 are projection views illustrating an auxiliary unit shown in FIG. 1;

FIG. 4A is an image showing a state in which a mat shown in FIG. 1 is folded and an air massage module is exposed to the outside;

FIG. 4B is an image showing a state in which first and second air pockets shown in FIG. 3 are disposed at both sides of a fixing portion;

FIG. 5 is a cross-sectional view taken along line A-A shown in FIG. 3;

FIG. 6A is a perspective image showing a mat in a thermotherapy bed according to a second exemplary embodiment of the present invention;

FIG. 6B is an image showing a state in which the mat shown in FIG. 6A is folded and an air massage module is exposed to the outside;

FIG. 7A is an image showing an extension gender;

FIG. 7B is an image showing a state in which the extension gender shown in FIG. 7A is attached to an air pocket; and

FIGS. 8A and 8B are images showing a state in which an arrangement position of an air massage module is adjusted from the mat according to a position of a user's calves.

### DETAILED DESCRIPTION OF THE INVENTION

Exemplary embodiments of the present invention are described with reference to the accompanying drawings in order to describe the present invention in detail so that those having ordinary knowledge in the technical field to which the present invention pertains can easily practice the present invention.

It should be noted that the same reference numerals are used to designate the same or similar elements throughout the drawings.

Further, in describing the present invention, well-known functions or constructions will not be described in detail since they may unnecessarily obscure the understanding of the present invention.

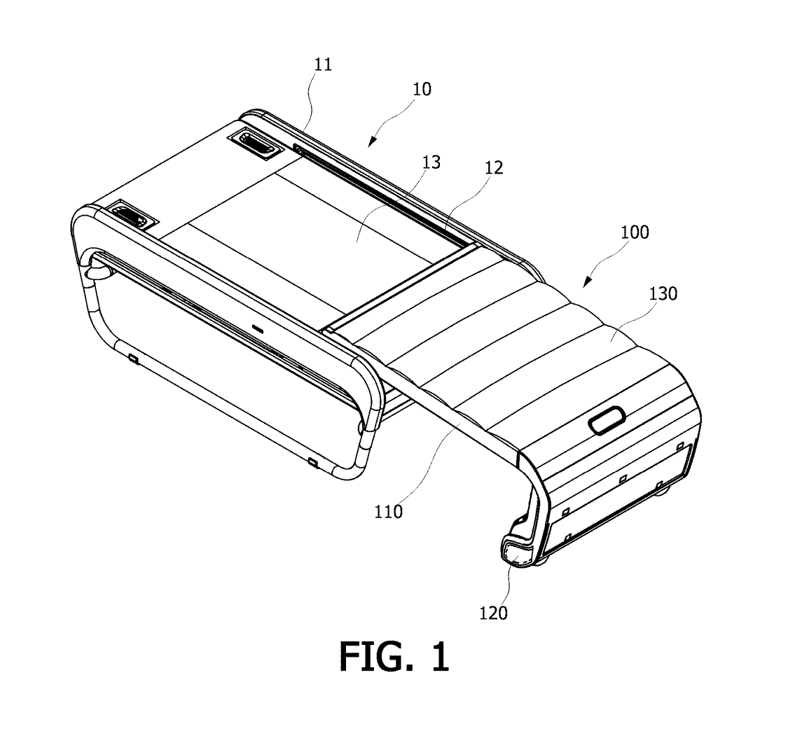

Prior to detailed descriptions of the present invention, in directions in the following descriptions, the term “upper” of “upper portion” is defined as a direction from a base portion 110 to a mat 130 shown in FIG. 1, and the term “lower” of “lower portion” is defined as a direction from the mat 130 to the base portion 110.

Hereinafter, a thermotherapy bed according to a first exemplary embodiment of the present invention will be described with reference to FIGS. 1 to 8.

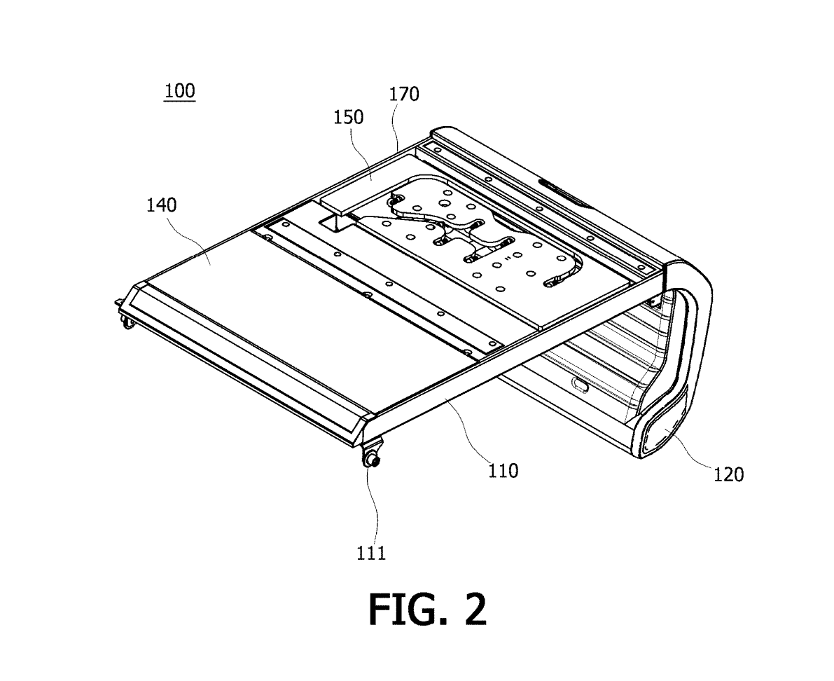

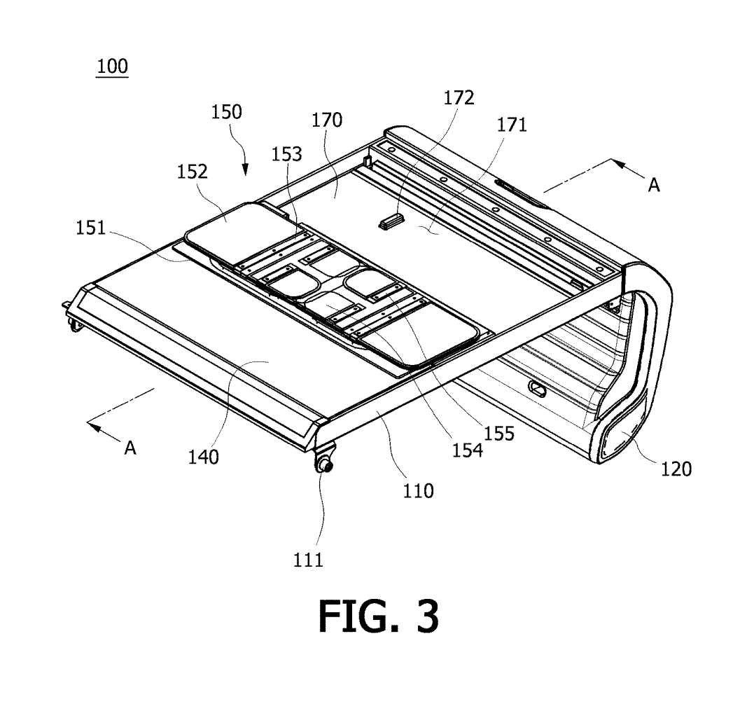

FIG. 1 is a perspective view illustrating the thermotherapy bed according to the first exemplary embodiment of the present invention, and FIGS. 2 and 3 are projection views illustrating an auxiliary unit 100 shown in FIG. 1.

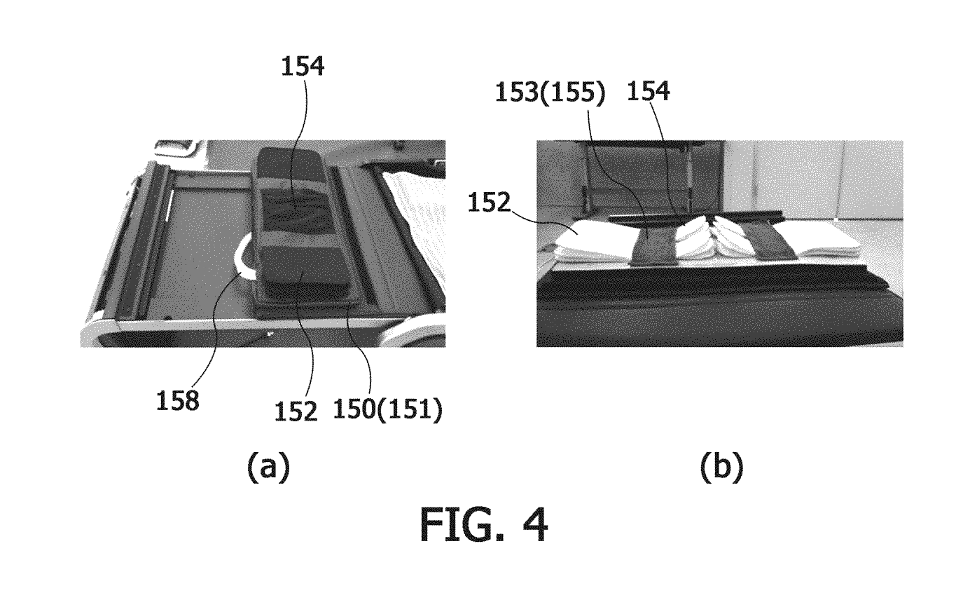

FIG. 4A is an image showing a state in which the mat 130 shown in FIG. 1 is folded and an air massage module 150 is exposed to the outside, and FIG. 4B is an image showing a state in which first and second air pockets 152 and 154 shown in FIG. 3 are disposed at both sides of a fixing portion.

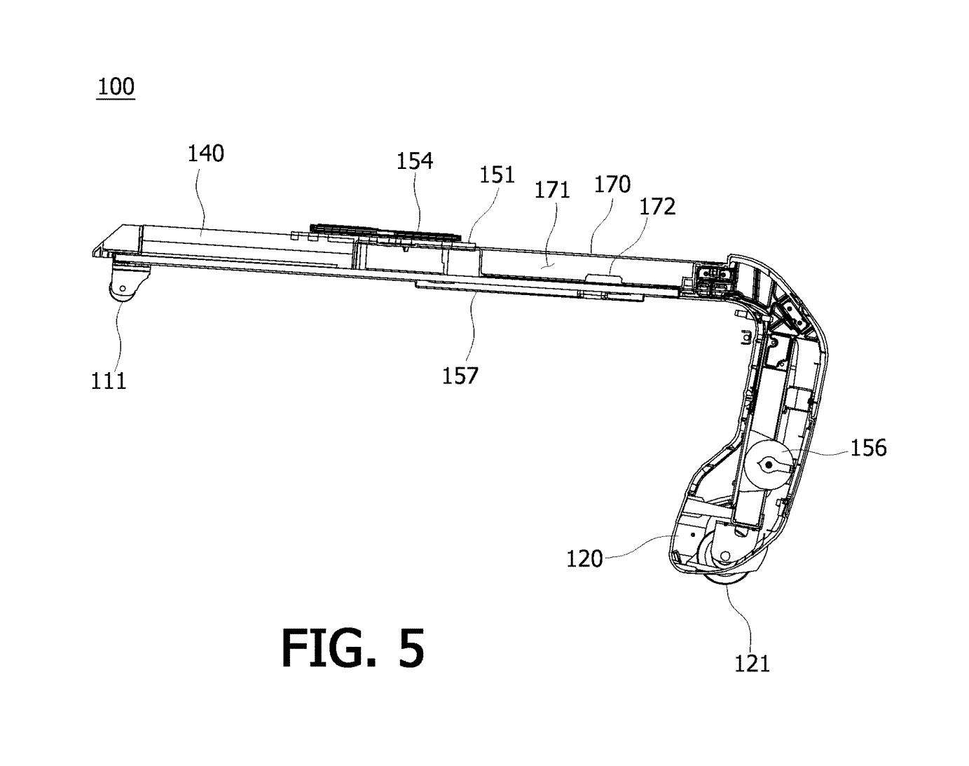

FIG. 5 is a cross-sectional view taken along line A-A shown in FIG. 3.

Referring to FIGS. 1 to 3, the thermotherapy bed includes a body 10, the auxiliary unit 100, the mat 130, and the air massage module 150.

A thermotherapy device 13 is installed in the body 10. The auxiliary unit 100 slides on the body 10 to be unfolded or to overlap the body 10, and thus, the thermotherapy device 13 is opened or closed.

A frame 11 is installed in the body 10 to support the thermotherapy device 13.

Guide grooves 12 are formed between the thermotherapy device 13 and the frame 11 in a lengthwise direction of the frame 11, and the auxiliary unit 100 slides along the guide grooves 12.

The auxiliary unit 100 includes a base portion 110 and a movement portion 120.

The base portion 110 slides on an upper portion of the body 10, and the movement portion 120 extends downward from an end of one side of the base portion 110.

Guide protrusions 111 mounted to the guide grooves 12 are installed on both side surfaces at an end of the other side of the base portion 110.

The guide protrusions 111 are moved along the guide grooves 12, and thus, the base portion 110 slides on the upper portion of the body 10.

A moving wheel 121 is installed at an end of the movement portion 120 and assists a sliding movement of the base portion 110.

A thermotherapy module 140 and the air massage module 150 are installed in the base portion 110.

Therefore, when the auxiliary unit 100 slides to overlap the body 10, the thermotherapy bed may be used as a heating sofa (stool).

In addition, when the auxiliary unit 100 slides from the body 10 and the thermotherapy bed is used as a heat massage device, a user may select and use the air massage module 150 and the thermotherapy module 140 of the auxiliary unit 100.

More specifically, the thermotherapy module 140 is installed at the other side of the base portion 110, at which the guide protrusions 111 are formed.

The air massage module 150 is configured to massage user's calves, is installed in the mat 130, and is stored in the auxiliary unit 100.

The base portion 110 has a module storage portion 170 for storing the air massage module 150.

The module storage portion 170 has a space portion 171 for storing the air massage module 150.

The air massage module 150 is wrapped by a finishing material such as leather or a cloth. The mat 130 is partially folded, so that the air massage module 150 is rotated.

That is, the air massage module 150 may be disposed on an upper portion of the base portion 110 or may be stored in the module storage portion 170 in accordance with a position of a user's calves by partially folding the mat 130.

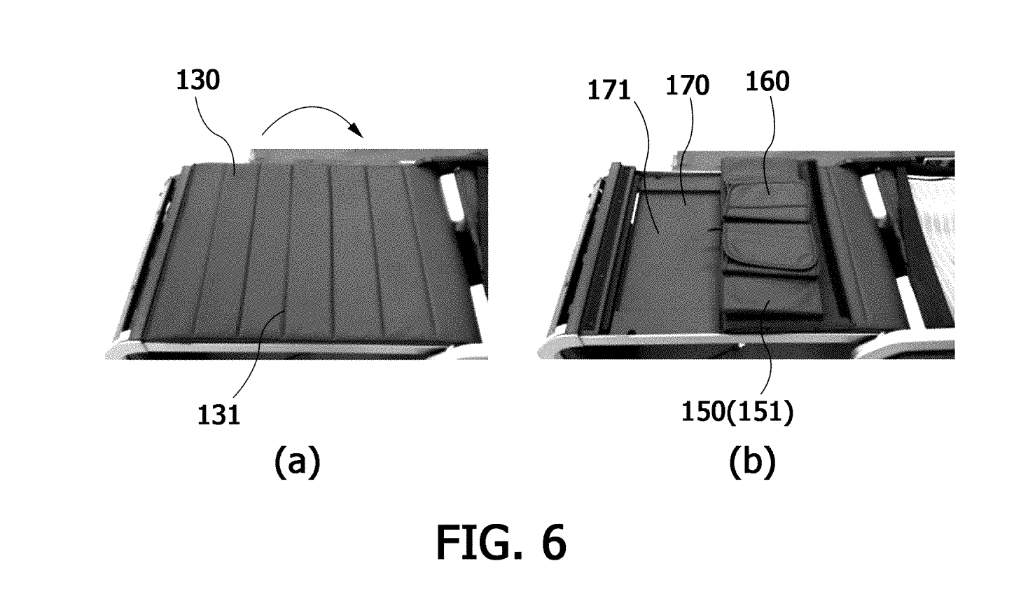

Here, in consideration of the position of the user's calves and in order to further facilitate rotation of the mat 130, the air massage module 150 may be rotated in a direction perpendicular to folding lines 131 formed in the mat 130 (shown in FIG. 6A).

A plurality of folding lines 131 are sequentially formed in the mat 130 and are spaced apart from each other by a certain distance.

That is, the air massage module 150 may be rotated in the direction perpendicular to the folding lines 131 and thus be disposed on the upper portion of the base portion 110 or be stored in the module storage portion 170 in accordance with a position of the user's calves.

Referring to FIGS. 3 to 5, the air massage module 150 includes a plate portion 151 and air pockets.

The air massage module 150 includes an air pump 156 configured to supply air to the air pockets and a hose 158 through which air supplied from the air pump 156 flows.

The plate portion 151 is configured to install the air pockets and also has a function for preventing the air pockets from being exposed to the outside when the air massage module 150 is stored in the module storage portion 170.

The air pockets have the first air pocket 152 and the second air pocket 154.

The first and second air pockets 152 and 154 include a plurality of air cells which are vertically stacked and are fixed to the plate portion 151 by first and second fixing portions 153 and 155.

More specifically, the first air pocket 152 is fixed to one side of the first fixing portion 153, and the second air pocket 154 is fixed to the other side of the first fixing portion 153 with respect to the first fixing portion 153.

One pair of first air pockets 152 are provided and are respectively fixed to the first and second fixing portions 153 and 155. Two pairs of second air pockets 154 are provided and are respectively fixed to the first and second fixing portions 153 and 154.

Here, one pair of second air pockets 154 may be provided as in the first air pockets 152.

The air pockets are expanded into a fan shape with respect to the first and second fixing portions 153 and 154 by the air supplied from the air pump 156 and perform a massage by surrounding the user's calves.

In order to confirm a storage state of the air massage module 150, a sensor 172 is installed on a lower portion of the module storage portion 170, more specifically, on a lower inner surface, and a magnet is installed in the plate portion 151.

The sensor 172 is a magnetoresistive (MR) sensor. The sensor 172 may measure a magnetic field intensity of the magnet to confirm whether the air massage module 150 is in a state of being stored in the module storage portion 170 or is in a state of being carried out of the module storage portion 170.

That is, the sensor 172 may confirm a storage state of the air massage module 150 by comparing a magnetic field intensity of the magnet in a state in which the air massage module 150 is stored in the module storage portion 170 with a magnetic field intensity of the magnet in a state in which the air massage module 150 is exposed to the outside from the module storage portion 170.

The sensor 172 is not limited to the MR sensor. The sensor 172 may be replaced with a light sensor, a mechanical push switch, a photo coupler, or the like.

A hose arrangement portion 157, in which the hose 158 is accommodated, is formed in a lower portion of the base portion 110. The movement portion 120 has a pump storage portion in which the air pump 156 is accommodated.

That is, the air pump 156 and the hose 158 may be accommodated inside the thermotherapy bed, thereby obtaining an effect of enhanced aesthetic sense.

Here, the formation position of the hose arrangement portion 157 is not limited to the lower portion of the base portion 110 and the hose arrangement portion 157 may also be formed in an upper portion of the base portion 110.

Hereinafter, a thermotherapy bed according to a second exemplary embodiment of the present invention will be described with reference to FIGS. 6A to 8B.

Prior to description, redundant description of the same components as the above-described first exemplary embodiment of the present invention will be omitted.

FIG. 6A is a perspective image showing a mat 130 in the thermotherapy bed according to the second exemplary embodiment of the present invention, and FIG. 6B is an image showing a state in which the mat 130 shown in FIG. 6A is folded and an air massage module 150 is exposed to the outside.

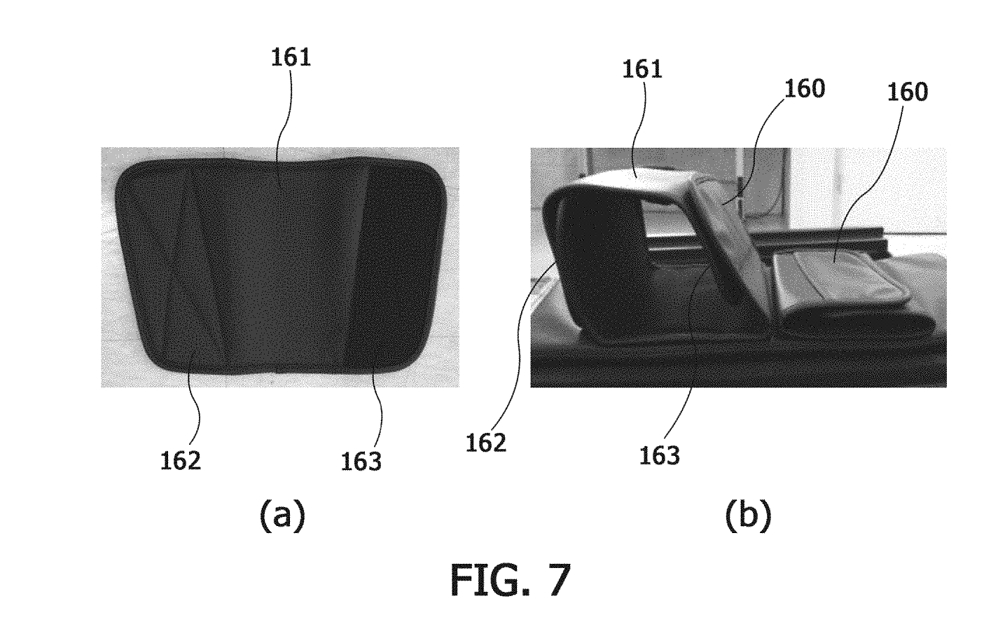

FIG. 7A is an image showing an extension gender, and FIG. 7B is an image showing a state in which the extension gender shown in FIG. 7A is attached to an air pocket 160.

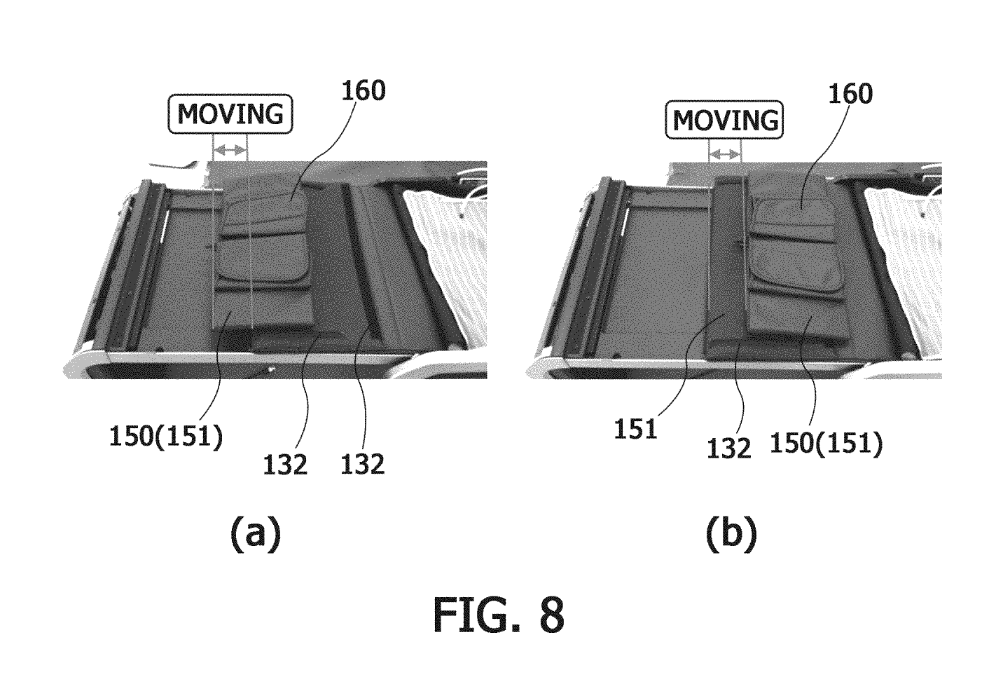

FIGS. 8A and 8B are images showing a state in which an arrangement position of the air massage module 150 is adjusted from the mat 130 according to a position of a user's calves.

The thermotherapy bed according to the second exemplary embodiment of the present invention includes one pair of air pockets 160 installed on the plate portion 151 to surround the user's calves.

Detachable portions are formed at ends of both sides of the air pocket 160 and have a female portion and a male portion, which are made of a Velcro material.

In consideration of different calf circumference lengths according to users, the air pocket 160 includes the extension gender attached to and detached from the detachable portions.

The extension gender has a cover portion 161 which surrounds the user's calf and has detachable portions (female and male portions) 162 and 163 formed at the ends of both sides of the cover portion 161.

That is, a circumference length of the air pocket 160 may be extended by attaching the female portion 162 and the male portion 163 of the detachable portions formed in the extension gender to the detachable portions formed in the air pocket 160.

As described above, since a position of the calves varies according to heights of the users, the air massage module 150 may be disposed at a position of the user's calves by rotating the air massage module 150 in the direction perpendicular to the folding lines 131 of the mat 130.

Here, in order to more precisely adjust an arrangement position of the air massage module 150, detachable portions 132 are formed in the mat 130 and the plate portion 151, so that it is possible to adjust the arrangement position of the air massage module 150.

As shown in FIGS. 8A and 8B, on the basis of a state in which the air massage module 150 is exposed to the outside, one or more detachable portions 132 are formed on a lower edge of the plate portion 151, and the detachable portions 132 are formed at positions of an upper portion of the mat 130, which correspond to the detachable portions 132 formed on the plate portion 151.

That is, a female portion and a male portion of the detachable portions 132 formed on the mat 130 may be attached to and detached from the detachable portions 132 formed on the plate portion 151, thereby more precisely adjusting the arrangement position of the air massage module 150 according to a position of a user's calves.

As described above, according to a thermotherapy bed according to various exemplary embodiments of the present invention, an arrangement space may be further secured through the auxiliary unit 100 sliding on the body 10 to be unfolded or to overlap the body 10.

In addition, when the auxiliary unit 100 slides to overlap the body 10, the thermotherapy bed may be used as a heating sofa (stool).

Further, when the auxiliary unit 100 slides from the body 10 to be unfolded and the thermotherapy bed is used as a heat massage device, the user may select and use the air massage module 150 and the thermotherapy module 140 of the auxiliary unit 100.

Furthermore, a calf massage may be easily performed through the air massage module 150 and the module storage portion 170, and an easy storage effect may be obtained.

In addition, the air pump 156 and the hose 158 can be accommodated inside the thermotherapy bed, thereby obtaining an effect of enhanced aesthetic sense.

Furthermore, in consideration of different positions of calves according to the users, an arrangement position of the air massage module 150 may be adjusted.

As described above, optimal embodiments of the present invention have been disclosed in the drawings and the specification. Although specific terms have been used in the present specification, these are merely intended to describe the present invention, and are not intended to limit the meanings thereof or the scope of the present invention described in the accompanying claims. Therefore, those skilled in the art will appreciate that various modifications and other equivalent embodiments are possible from the embodiments. Therefore, the technical scope of the present invention should be defined by the technical spirit of the claims.

1. A thermotherapy bed comprising:
a body in which a thermotherapy device is installed;
an auxiliary unit configured to slide on the body to be unfolded or to overlap the body, such that the thermotherapy device is opened or closed;
a mat configured to cover an upper portion of the auxiliary unit; and
an air massage module installed in the mat and stored in the auxiliary unit.

2. The thermotherapy bed of claim 1, wherein the auxiliary unit has a base portion sliding on an upper portion of the body, and
a movement portion extending downward from an end of one side of the base portion.

3. The thermotherapy bed of claim 2, wherein the base portion has a module storage portion for storing the air massage module.

4. The thermotherapy bed of claim 3, wherein the module storage portion includes a sensor configured to determine whether the air massage module is stored.

5. The thermotherapy bed of claim 3, wherein the air massage module is stored in the module storage portion and is not exposed to the outside, and subsequent to when a user uses the thermotherapy bed, the mat is partially folded, such that the air massage module is exposed to the outside from the module storage portion.

6. The thermotherapy bed of claim 1, wherein the air massage module includes a plate portion installed on a lower portion of the mat and air pockets installed on the plate portion.

7. The thermotherapy bed of claim 6, wherein the air pockets have first and second air pockets disposed at both sides of a fixing portion installed on the plate portion.

8. The thermotherapy bed of claim 7, wherein the first and second air pockets include a plurality of air cells which are vertically stacked.

9. The thermotherapy bed of claim 7, wherein a user's calves are disposed at the fixing portion, and
the first and second air pockets are expanded into a fan shape with respect to the fixing portion by air supplied from an air pump and surround the user's calves.

10. The thermotherapy bed of claim 6, wherein detachable portions are formed at ends of both sides of the air pocket.

11. The thermotherapy bed of claim 10, wherein the air pocket includes an extension gender attached to and detached from the detachable portions.
